(19) United States
(12) Patent Application Publication
(10) Pub. No.: US 2025/0340719 A1
(43) Pub. Date: Nov. 6, 2025

(54) CROSSLINKING AGENT FOR CHLORINATED BUTYL RUBBER

(71) Applicant: KAWAGUCHI CHEMICAL INDUSTRY CO., LTD., Tokyo (JP)

(72) Inventors: Takeshi OHNUKI, Kawaguchi-shi (JP); Youhei UTSUGI, Kawaguchi-shi (JP); Yasuyuki HIRUNUMA, Kawaguchi-shi (JP); Anzu SAITO, Kawaguchi-shi (JP); Kazuo TSUJIMOTO, Kawaguchi-shi (JP)

(73) Assignee: KAWAGUCHI CHEMICAL INDUSTRY CO., LTD., Tokyo (JP)

(21) Appl. No.: 18/868,971

(22) PCT Filed: Jul. 3, 2023
(86) PCT No.: PCT/JP2023/024647

§ 371 Date: Nov. 25, 2024

(30) Foreign Application Priority Data
Jul. 8, 2022 (JP) 2022-12004

(51) Int. Cl. C08K 5/378; C07D 251/46; C08J 3/24

(52) U.S. Cl. CPC C08K 5/378; C07D 251/46; C08J 3/24; C08J 2323/28

(57) ABSTRACT

There is provided a crosslinking agent for chlorinated butyl rubber that allows for a fast crosslinking reaction rate and excellent rubber elasticity. With the use of a primary aminotriazine dithiol compound represented by Formula (1) below as a crosslinking agent for a chlorinated butyl rubber composition, it becomes possible to crosslink the composition at a faster rate and to obtain crosslinked rubber with excellent rubber elasticity.

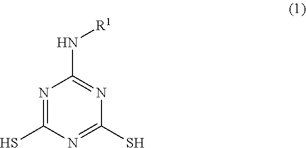

where R¹ is a linear or branched hydrocarbon group having 6 to 10 carbon atoms.

### TECHNICAL FIELD

The present invention relates to a crosslinking agent for chlorinated butyl rubber and, more specifically, to a crosslinking agent suitable for use in the production of medical rubber products made from a chlorinated butyl rubber composition.

### BACKGROUND ART

Halogenated butyl rubber, which has excellent impermeability to gases, liquids and solids (i.e., three states of matter), is a rubber material for tubes, packing, sealing, and the like.

With such properties, halogenated butyl rubber is also suitable as a material for medical rubber products, such as rubber stoppers for vials and gaskets for syringes.

Halogenated butyl rubber is classified into chlorinated butyl rubber and brominated butyl rubber, which are different in properties.

For example, brominated butyl rubber (hereinafter, abbreviated as BIIR) has high crosslinking reactivity, so that it can be crosslinked in a short time. Thus, BIIR is superior in productivity to chlorinated butyl rubber (hereinafter abbreviated as CIIR). Meanwhile, since BIIR contains additives such as epoxidized soybean oil as a stabilizer, elution in water (hereinafter, “elution in water” will be referred to simply as “elution”) of such additives may be problematic.

On the other hand, CIIR, which is inferior in productivity to BIIR, is superior to BIIR in suppressing elution of additives, as it requires no stabilizer.

Halogenated butyl rubber can be crosslinked by several methods, such as sulfur crosslinking, peroxide crosslinking, metal oxide crosslinking, resin crosslinking, amine crosslinking, and thiol crosslinking, depending on what type of crosslinking agent is used. For medical purposes where biosafety needs to be ensured, thiol crosslinking is commonly used because it causes less elution of a crosslinking agent. Note that the crosslinking agent is one of the additives for use in halogenated butyl rubber.

Specific examples of the crosslinking agent for use in thiol crosslinking include aminotriazine dithiol compounds such as, in particular, 6-(di-n-butylamino)-1,3,5-triazine-2,4-dithiol (hereinafter, abbreviated as “BSH”), which is commercially available and is commonly used (Patent Document 1).

In view of the above, CIIR has conventionally been used with BSH as a crosslinking agent for the production of medical rubber products, such as rubber stoppers and gaskets, especially in cases where less elution of additives is required.

However, such an extremely excellent combination of CIIR with BSH that causes less elution of additives has low productivity because of its slow crosslinking reaction rate. As such, there has been a demand for a crosslinking agent that allows for a faster crosslinking reaction rate.

In order to respond to recent strong demand for medical rubber products with biosafety ensured, it is required to provide a crosslinking agent that causes less elution of additives and allows for a faster crosslinking reaction rate.

Prior to the present invention, 6-[bis(2-ethylhexyl)amino]-1,3,5-triazine-2,4-dithiol (Patent Document 2) was known, which is an aminotriazine dithiol compound as a crosslinking agent that allows for a faster crosslinking reaction rate than BSH. However, this compound has a higher molecular weight than BSH and, thus, needs to be contained in a larger amount to ensure enough rubber elasticity required of rubber stoppers and gaskets.

Increasing the content of the crosslinking agent could lead to an increase in the cost of producing products such as rubber stoppers and gaskets. Thus, it is required to provide a new crosslinking agent that allows for a fast crosslinking reaction rate and ensures rubber elasticity for a crosslinked rubber product at a level equal to or higher than that achieved by BSH even when the agent is contained in an amount almost equal to or smaller than BSH.

### PRIOR ART DOCUMENTS

### Patent Documents

Patent Document 1: JP-A-2002-301133

Patent Document 2: JP-A-2014-237797

### SUMMARY OF THE INVENTION

### Problems to be Solved by the Invention

In order to solve the above-described problems, the present inventors have conducted intensive studies and consequently completed the present invention, which relates to an aminotriazine dithiol compound represented by Formula (1) below for use as a crosslinking agent. With the use of this compound, it becomes possible both to crosslink a chlorinated butyl rubber composition at a fast rate and to give high rubber elasticity.

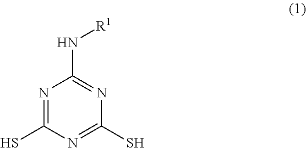

where R¹ is a linear or branched hydrocarbon group having 6 to 10 carbon atoms.

In light of the foregoing, the present invention provides a crosslinking agent for chlorinated butyl rubber that allows for a fast crosslinking reaction rate and excellent rubber elasticity.

### Means for Solving the Problems

The present invention provides a crosslinking agent for chlorinated butyl rubber represented by Formula (1) below:

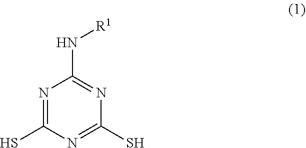

where R¹ is a linear or branched hydrocarbon group having 6 to 10 carbon atoms.

The rubber composition preferably includes a compound represented by Formula (1) in an amount of 0.01 to 10 parts by weight per 100 parts by weight of chlorinated butyl rubber.

A rubber product obtained by crosslinking the rubber composition is preferable.

A medical rubber composition preferably includes the rubber composition.

A medical rubber product obtained by crosslinking the medical rubber composition is preferable.

### Effect of the Invention

According to the present invention, an aminotriazine dithiol compound represented by Formula (1) above is used as a crosslinking agent for a chlorinated butyl rubber composition. As a result, a highly elastic medical rubber product that causes less elution of additives can be produced with improved productivity.

### BRIEF DESCRIPTION OF THE DRAWINGS

FIG. 1 is a diagram showing the relationship between vulcanization time and rubber elasticity in Examples and Comparative Examples.

### MODE FOR CARRYING OUT THE INVENTION

Hereinafter, the present invention will be described by way of specific embodiments.

The base rubber component in the present invention is chlorinated butyl rubber (CIIR), which can be used alone, or alternatively in combination with other rubber components such as brominated butyl rubber (BIIR), synthetic polyisoprene rubber (IR), styrene butadiene copolymer rubber (SBR), acrylonitrile butadiene rubber (NBR), ethylene propylene diene copolymer rubber (EPDM), and polyisobutylene rubber (IIR) so that the properties of such rubbers may be imparted. The base rubber component CIIR, when used in combination with other rubber components, is used in an amount of at least 50 parts by weight or more, preferably 80 parts by weight or more, and more preferably 95 parts by weight or more, based on the total rubber component taken as 100 parts by weight.

The crosslinking agent of the present invention is an aminotriazine dithiol compound represented by Formula (1) above, and is used in an amount of 0.01 to 10 parts by weight, preferably 0.5 to 5 parts by weight, per 100 parts by weight of the chlorinated butyl rubber.

The aminotriazine dithiol compound represented by Formula (1) above can be used alone or optionally in combination with other crosslinking agents. Other crosslinking agents may be used in combination to replace 50% by weight or less, preferably 30% by weight or less, and more preferably 10% by weight or less, of the aminotriazine dithiol compound represented by Formula (1) taken as 100% by weight.

If the linear or branched hydrocarbon group in R¹ of the aminotriazine dithiol compound represented by Formula (1) has 5 or less carbon atoms, which is outside the range of the present invention, such an aminotriazine dithiol compound also serves to crosslink CIIR, but not at a sufficiently fast rate.

Moreover, if the linear or branched hydrocarbon group in R¹ has 11 or more carbon atoms, such an aminotriazine dithiol compound also serves to crosslink CIIR, but causes a decrease in rubber elasticity, so that sufficiently high rubber elasticity cannot be achieved.

Further, if an alicyclic or aromatic hydrocarbon group is attached to an aminotriazine dithiol compound for use as a crosslinking agent, such a compound neither achieves a sufficiently fast crosslinking rate nor ensures sufficiently high rubber elasticity, even when the hydrocarbon group in R¹ has 6 to 10 carbon atoms.

The primary aminotriazine dithiol compound specified in the present invention ensures both a fast crosslinking reaction rate and high rubber elasticity, and causes less elution of additives without serving as a source of nitrosamine, which is important for medical applications.

There is no particular limitation on the compounding agent required for a medical rubber composition mainly containing chlorinated butyl rubber. For example, even an existing rubber composition containing BSH as a crosslinking agent can also exhibit the effect of the present invention simply by replacing the BSH with the primary aminotriazine dithiol compound specified in the present invention.

### EXAMPLES

Hereinafter, the present invention will be described in more detail with reference to, but not limited to, Examples.

### Example 1: Synthesis of 6-(n-hexylamino)-1,3,5-triazine-2,4-dithiol (1-1)

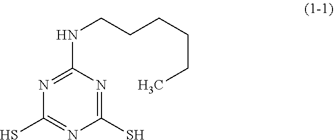

90.2 g (489 mmol) of cyanuric chloride and 165 g of toluene were placed in a flask and cooled to 5° C. or lower, followed by stirring. To this mixture, 49.6 g (490 mmol) of hexylamine (having 6 carbon atoms and a linear structure) dissolved in 165 g of toluene was added dropwise at 20° C. or lower, followed by stirring at the same temperature for two hours. Thereafter, an aqueous solution of sodium hydroxide was added, followed by separation. The resultant organic layer was warmed to 50° C. while being stirred, to which an aqueous solution of sodium hydrosulfide was added dropwise, followed by stirring for one hour. To this mixture, 30 wt % sulfuric acid was added dropwise. The resulting suspension were filtered, washed, and dried at 80° C., thereby obtaining 96.0 g of a desired white crystal.

The following is the analysis for the structural identification of this compound:

¹H-NMR (solvent: DMSO-d₆)

### 0.86 (t, J=6.8 Hz, 3H), 1.26-1.28 (m, 6H), 1.46-1.47 (m, 2H), 3.27-3.31 (m, 2H), 7.11 (br, 1H), 12.22 (br, 1H), 12.87 (s, 1H)

¹³C-NMR (solvent: DMSO-d₆)

13.9, 22.1, 25.8, 28.5, 30.9, 40.4, 149.8, 173.7, 182.9

### Example 2: Synthesis of 6-(n-heptylamino)-1,3,5-triazine-2,4-dithiol (1-2)

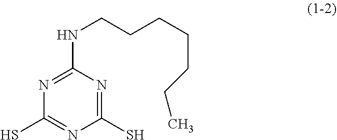 [SMILES: CCCCCCCNc1nc(S)nc(S)n1]

107 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 56.5 g (490 mmol) of heptylamine (having 7 carbon atoms and a linear structure).

The following is the analysis for the structural identification of this compound:

¹H-NMR (solvent: DMSO-d₆)

0.85 (t, J=6.9 Hz, 3H), 1.26 (m, 8H), 1.46-1.49 (m, 2H), 3.27-3.31 (m, 2H), 7.11 (br, 1H), 12.22 (br, 1H), 12.88 (s, 1H)

¹³C-NMR (solvent: DMSO-d₆)

14.2, 22.2, 26.3, 28.5, 28.7, 31.4, 40.5, 150.0, 174.0, 182.9

### Example 3: Synthesis of 6-(n-octylamino)-1,3,5-triazine-2,4-dithiol (1-3)

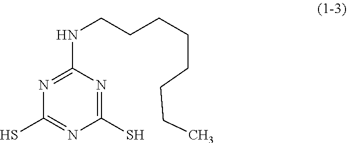 [SMILES: CCCCCCCCNc1nc(S)nc(S)n1]

84.5 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 63.8 g (494 mmol) of octylamine (having 8 carbon atoms and a linear structure).

The following is the analysis for the structural identification of this compound:

¹H-NMR (solvent: DMSO-d₆)

0.86 (t, J=7.0 Hz, 3H), 1.26 (m, 10H), 2.04 (m, 2H), 3.29 (m, 2H), 7.12 (br, 1H), 12.23 (br, 1H), 12.90 (s, 1H)

¹³C-NMR (solvent; DMSO-d₆)

14.0, 22.1, 26.1, 28.6, 28.7, 28.7, 31.3, 40.4, 149.8, 173.5, 183.1

### Example 4: Synthesis of 6-(2-ethylhexylamino)-1,3,5-triazine-2,4-dithiol (1-4)

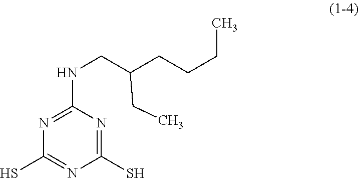 [SMILES: CCCCC(CC)CNc1nc(S)nc(S)n1]

120 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 64.3 g (497 mmol) of 2-ethylhexylamine (having 8 carbon atoms and a branched structure).

The following is the analysis for the structural identification of this compound:

¹H-NMR (solvent: DMSO-d₆)

0.85 (t, J=7.6 Hz, 3H), 0.87 (t, J=6.7 Hz, 3H), 1.25-1.29 (m, 8H), 1.51 (m, 1H), 3.27 (m, 2H), 7.04 (br, 1H), 12.03 (br, 1H), 12.90 (s, 1H)

¹³C-NMR (solvent: DMSO-d₆)

10.7, 14.0, 22.5, 23.5, 28.2, 30.1, 38.3, 43.0, 150.0, 173.2, 183.2

### Example 5; Synthesis of 6-(nonylamino)-1,3,5-triazine-2,4-dithiol (1-5)

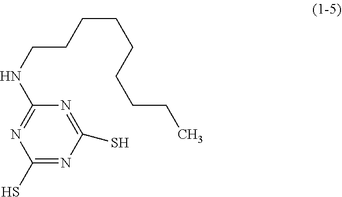 [SMILES: CCCCCCCCCNc1nc(S)nc(S)n1]

94.6 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 70.2 g (490 mmol) of nonylamine (having 9 carbon atoms and a linear structure).

The following is the analysis for the structural identification of this compound:

¹H-NMR (solvent: DMSO-d₆)

0.85 (t, J=6.9 Hz, 3H), 1.24-1.25 (m, 12H), 1.47-1.48 (m, 2H), 3.27-3.31 (m, 2H), 7.11 (br, 1H), 12.22 (br, 1H), 12.88 (s, 1H)

¹³C-NMR (solvent: DMSO-d₆)

14.2, 22.3, 26.3, 28.7, 28.9 (2C), 29.2, 31.5, 40.5, 150.0, 173.8, 183.1

### Example 6: Synthesis of 6-(decylamino)-1,3,5-triazine-2,4-dithiol (1-6)

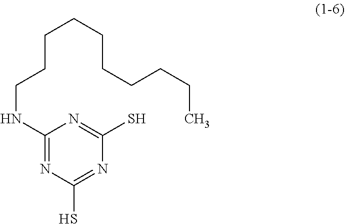 [SMILES: CCCCCCCCCCNc1nc(S)nc(S)n1]

101 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 77.1 g (490 mmol) of decylamine (having 10 carbon atoms and a linear structure).

The following is the analysis for the structural identification of this compound:

¹H-NMR (solvent: DMSO-d₆)

0.85 (t, J=7.0 Hz, 3H), 1.25-1.26 (m, 14H), 1.48-1.49 (m, 2H), 3.27-3.31 (m, 2H), 7.12 (br, 1H), 12.24 (br, 1H), 12.90 (s, 1H)

¹³C-NMR (solvent: DMSO-d₆)

14.0, 22.2, 26.1, 28.6, 28.7, 28.8, 29.0, 29.0, 31.4, 40.3, 149.8, 173.7, 183.1

### Comparative Example 2: Synthesis of 6-(ethylamino)-1,3,5-triazine-2,4-dithiol (2-1)

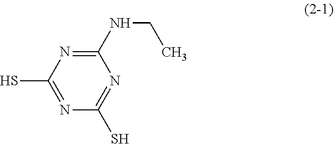

42.4 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 22.1 g (490 mmol) of ethylamine (having 2 carbon atoms and a linear structure).

The following is the analysis for the structural identification of this compound:

¹H-NMR (solvent: DMSO-d₆)

1.09 (t, J=7.2 Hz, 3H), 3.31-3.36 (m, 2H), 7.13 (br, 1H), 12.36 (br, 1H), 12.90 (s, 1H)

¹³C-NMR (solvent: DMSO-d₆)

14.6, 35.7, 149.8, 173.9, 183.1

### Comparative Example 3: Synthesis of 6-(n-butylamino)-1,3,5-triazine-2,4-dithiol (2-2)

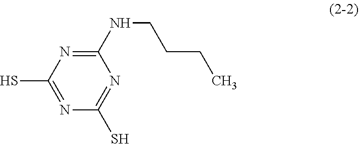

76.4 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 35.8 g (490 mmol) of butylamine (having 4 carbon atoms and a linear structure).

The following is the analysis for the structural identification of this compound:

¹H-NMR (solvent: DMSO-d₆)

0.88 (t, J=7.3 Hz, 3H), 1.29 (sext, J=7.4 Hz, 2H), 1.47 (quin, J=7.3 Hz, 2H), 3.28-3.32 (m, 2H), 7.12 (br, 1H), 12.22 (br, 1H), 12.87 (s, 1H)

¹³C-NMR (solvent; DMSO-d₆)

13.8, 19.5, 30.8, 40.2, 150.0, 174.1, 182.8

### Comparative Example 4: Synthesis of 6-(n-pentylamino)-1,3,5-triazine-2,4-dithiol (2-3)

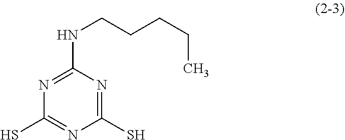

100 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 42.7 g (490 mmol) of pentylamine (having 5 carbon atoms and a linear structure).

The following is the analysis for the structural identification of this compound:

¹H-NMR (solvent: DMSO-d₆)

0.86 (t, J=7.1 Hz, 3H), 1.21-1.33 (m, 4H), 1.48 (quin, J=7.3 Hz, 2H), 3.27-3.31 (m, 2H), 7.11 (br, 1H), 12.22 (br, 1H), 12.88 (s, 1H)

¹³C-NMR (solvent: DMSO-d₆)

14.1, 22.0, 28.4, 28.5, 40.5, 149.9, 173.7, 183.4

### Comparative Example 5: Synthesis of 6-(n-dodecylamino)-1,3,5-triazine-2,4-dithiol (2-4)

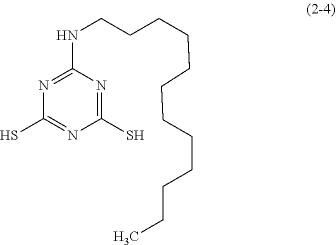 [SMILES: CCCCCCCCCCCCNc1nc(S)nc(S)n1]

82.8 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 90.7 g (490 mmol) of dodecylamine (having 12 carbon atoms and a linear structure).

The following is the analysis for the structural identification of this compound;

¹H-NMR (solvent: DMSO-d₆)

0.85 (t, J=7.0 Hz, 3H), 1.24-1.26 (m, 18H), 1.46-1.49 (m, 2H), 3.27-3.31 (m, 2H), 7.12 (br, 1H), 12.23 (br, 1H), 12.90 (s, 1H)

¹³C-NMR (solvent: DMSO-d₆)

14.0, 22.2, 26.1, 28.6, 28.7, 28.8, 29.0, 29.0, 29.1, 29.1, 31.4, 40.3, 149.8, 174.0, 183.0

### Comparative Example 6: Synthesis of 6-(cyclohexylamino)-1,3,5-triazine-2,4-dithiol (2-5)

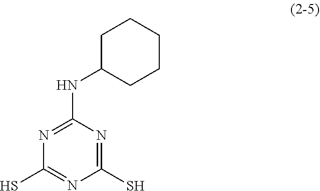 [SMILES: Sc1nc(S)nc(NC2CCCCC2)n1]

99.5 g of a desired white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 48.6 g (490 mmol) of cyclohexylamine (having 6 carbon atoms and an alicyclic structure).

The following is the analysis for the structural identification of this compound:

¹H-NMR (solvent: DMSO-d₆)

1.18-1.35 (m, 5H), 1.52-1.55 (m, 1H), 1.63-1.66 (m, 2H), 1.78-1.81 (m, 2H), 3.79-3.81 (m, 1H), 7.03 (br, 1H), 11.82 (br, 1H), 12.91 (s, 1H)

¹³C-NMR (solvent: DMSO-d₆)

24.2, 25.0, 32.0, 49.2, 149.2, 173.7, 183.3

### Comparative Example 7: Synthesis of 6-(cyclooctylamino)-1,3,5-triazine-2,4-dithiol (2-6)

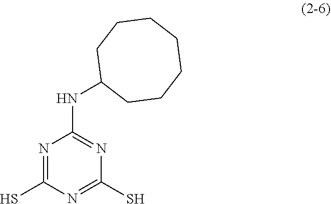 [SMILES: Sc1nc(S)nc(NC2CCCCCCC2)n1]

110 g of a desired slightly yellowish white crystal was obtained in a like manner except that hexylamine (having 6 carbon atoms and a linear structure) in the synthesis example of the compound 6-(n-hexylamino)-1,3,5-triazine-2,4-dithiol of Example 1 represented by Formula (1-1) was replaced with 62.3 g (490 mmol) of cyclooctylamine (having 8 carbon atoms and an alicyclic structure).

The following is the analysis for the structural identification of this compound:

¹H-NMR (solvent: DMSO-d₆)

1.48-1.60 (m, 12H), 1.76-1.77 (m, 2H), 3.98-4.03 (m, 1H), 7.07 (br, 1H), 11.73 (br, 1H), 12.90 (s, 1H)

¹³C-NMR (solvent: DMSO-d₆)

22.9, 24.8, 27.1, 30.9, 50.6, 148.9, 173.5, 183.1

Note that commercially available products were used for a compound of Comparative Example 1, which was 6-(di-n-butylamino)-1,3,5-triazine-2,4-dithiol (BSH), and a compound of Comparative Example 8, which was 6-(anilino)-1,3,5-triazine-2,4-dithiol (hereinafter, abbreviated as ASH; with a chemical structure having a phenyl group (having 6 carbon atoms and an aromatic structure) in place of the hexyl group (having 6 carbon atoms and a linear structure) in the compound of Example 1 represented by Formula (1-1)).

Next, a description will be given of test results for rubber obtained by using each of the compounds.

Table 1 shows materials of rubber compositions in Examples 1-6 and Comparative Examples 1-8. The component amount is expressed in parts by weight (phr). Examples 1-6 used the primary aminotriazine dithiol compounds of the present invention represented by Formulas (1-1)-(1-6), respectively. Comparative Example 1 used BSH, which is a commonly used conventional triazine crosslinking agent. Comparative Examples 2-7 used the primary aminotriazine dithiol compounds represented by Formulas (2-1)-(2-6), respectively. Comparative Example 8 used ASH.

Each of the rubber compositions was prepared by the usual kneading method using a closed mixer and an open roll mill. More specifically, chlorinated butyl rubber was mixed with chemicals, such as a filler, of Step A as shown in Table 1 and kneaded using a Banbury mixer. To this mixture, each of the crosslinking agents of Step B as shown in Table 1 was added using an open roll, thereby obtaining each of the rubber compositions.

TABLE 1-1

| | | Example 1 | Example 2 | Example 3 | Example 4 | Example 5 | Example 6 |
|---|---|---|---|---|---|---|---|
| Step A | Chlorinated butyl rubber (CIIR) | 100 | 100 | 100 | 100 | 100 | 100 |
| | Baked clay | 25 | 25 | 25 | 25 | 25 | 25 |
| | Titanium oxide | 5 | 5 | 5 | 5 | 5 | 5 |
| | N990 carbon black | 0.5 | 0.5 | 0.5 | 0.5 | 0.5 | 0.5 |
| | Magnesium oxide | 1 | 1 | 1 | 1 | 1 | 1 |
| | Stearic acid | 1 | 1 | 1 | 1 | 1 | 1 |
| Step B | Compound 2 | 2.0 | | | | | |
| | Compound 3 | | 2.0 | | | | |
| | Compound 4 | | | 2.0 | | | |
| | Compound 5 | | | | 2.0 | | |
| | Compound 6 | | | | | 2.0 | |
| | Compound 7 | | | | | | 2.0 |
| | Actor BSH¹⁾ | | | | | | |
| | Compound 8 | | | | | | |
| | Compound 9 | | | | | | |
| | Compound 10 | | | | | | |
| | Compound 11 | | | | | | |
| | Compound 12 | | | | | | |
| | Compound 13 | | | | | | |
| | Actor ASH²⁾ | | | | | | |

¹⁾6-(di-n-butylamino)-1,3,5-triazine-2,4-dithiol (BSH manufactured by Kawaguchi Chemical Industry Co., LTD.)
²⁾6-(anilino)-1,3,5-triazine-2,4-dithiol (ASH manufactured by Kawaguchi Chemical Industry Co., LTD.)

TABLE 1-2

| | | Comparative | Comparative | Comparative | Comparative | Comparative | Comparative | Comparative | Comparative |
|---|---|---|---|---|---|---|---|---|---|
| | | Example 1 | Example 2 | Example 3 | Example 4 | Example 5 | Example 6 | Example 7 | Example 8 |
| Step A | Chlorinated butyl | 100 | 100 | 100 | 100 | 100 | 100 | 100 | 100 |
| | rubber (CIIR) | | | | | | | | |
| | Baked clay | 25 | 25 | 25 | 25 | 25 | 25 | 25 | 25 |
| | Titanium oxide | 5 | 5 | 5 | 5 | 5 | 5 | 5 | 5 |
| | N990 carbon black | 0.5 | 0.5 | 0.5 | 0.5 | 0.5 | 0.5 | 0.5 | 0.5 |
| | Magnesium oxide | 1 | 1 | 1 | 1 | 1 | 1 | 1 | 1 |
| | Stearic acid | 1 | 1 | | | 1 | 1 | | 1 |
| Step B | Compound 2 | | | | | | | | |
| | Compound 3 | | | | | | | | |
| | Compound 4 | | | | | | | | |
| | Compound 5 | | | | | | | | |
| | Compound 6 | | | | | | | | |
| | Compound 7 | | | | | | | | |
| | Actor BSH¹⁾ | 2.0 | | | | | | | |
| | Compound 8 | | 2.0 | | | | | | |
| | Compound 9 | | | 2.0 | | | | | |
| | Compound 10 | | | | 2.0 | | | | |
| | Compound 11 | | | | | 2.0 | | | |
| | Compound 12 | | | | | | 2.0 | | |
| | Compound 13 | | | | | | | 2.0 | |
| | Actor ASH²⁾ | | | | | | | | 2.0 |

¹⁾6-(di-n-butylamino)-1,3,5-triazine-2,4-dithiol (BSH manufactured by Kawaguchi Chemical Industry Co., LTD.)
²⁾6-(anilino)-1,3,5-triazine-2,4-dithiol (ASH manufactured by Kawaguchi Chemical Industry Co., LTD.)

Each of the compositions thus obtained was subjected to a vulcanization test using an oscillating vulcanization tester (rheometer tester) in accordance with JIS K6300-2.

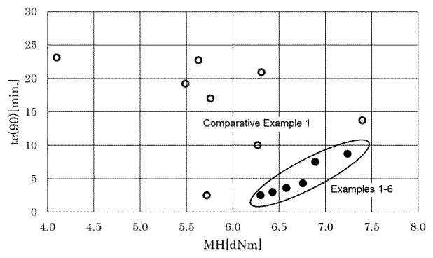

Table 2 shows ML (minimum elastic torque), MH (maximum elastic torque), tc10 (time required for 10% completion of vulcanization), and tc90 (time required for 90% completion of vulcanization), when the test was conducted at a temperature of 175° C. for 30 minutes. Further, FIG. 1 shows MH on the horizontal axis and tc90 on the vertical axis. As used herein, MH and tc90 served to indicate rubber elasticity and crosslinking reaction rate, respectively.

TABLE 2-1

| | Example 1 | Example 2 | Example3 | Example 4 | Example 5 | Example 6 |
|---|---|---|---|---|---|---|
| ML [dNm] | 1.4 | 1.4 | 1.4 | 1.5 | 1.4 | 1.4 |
| MH [dNm] | 7.2 | 6.9 | 6.6 | 6.8 | 6.4 | 6.3 |
| Tc (10) [min.] | 0.7 | 0.7 | 0.5 | 0.6 | 0.5 | 0.5 |
| Tc (90) [min.] | 8.7 | 7.5 | 3.6 | 4.3 | 3.0 | 2.5 |

TABLE 2-2

| | Comparative | Comparative | Comparative | Comparative | Comparative | Comparative | Comparative | Comparative |
|---|---|---|---|---|---|---|---|---|
| | Example 1 | Example 2 | Example3 | Example 4 | Example 5 | Example 6 | Example 7 | Example 8 |
| ML [dNm] | 1.2 | 1.3 | 1.3 | 1.4 | 1.4 | 1.3 | 1.3 | 1.3 |
| MH [dNm] | 6.3 | 5.6 | 6.3 | 7.4 | 5.7 | 5.5 | 5.8 | 4.1 |
| Tc (10) [min.] | 1.0 | 1.1 | 1.2 | 0.8 | 0.5 | 0.9 | 0.8 | 1.6 |
| Tc (90) [min.] | 10.0 | 22.7 | 20.9 | 13.7 | 2.5 | 19.2 | 17.0 | 23.1 |

As shown in Table 2 and FIG. 1, tc90 was shorter and MH was higher in Examples 1-6 than in Comparative Example 1 using a conventional triazine-based crosslinking agent. This indicates that the compositions of Examples 1-6 were able to be crosslinked in a short time and the resultant rubber had high elasticity.

In each of Comparative Examples 2-4, where the aminotriazine dithiol compound had a linear or branched hydrocarbon group having 5 or less carbon atoms at the position of R¹ in the aminotriazine dithiol compound represented by Formula (1), tc90 was longer than that in Comparative Example 1, which is an indication of a slow crosslinking reaction rate. In Comparative Example 5, where the compound had a linear or branched hydrocarbon group having 11 or more carbon atoms at the position of R¹, tc90 was shorter than that in Comparative Example 1, which is an indication of a fast crosslinking reaction rate. However, MH was 5.7 relative to 6.3 in Comparative Example 1, which is a clear indication of low rubber elasticity. In each of Comparative Examples 6 to 8, where the compound had an alicyclic or aromatic hydrocarbon group having 6 to 10 carbon atoms at the position of R¹, tc90 was longer and MH was lower than those in Comparative Example 1, which is an indication of a slow crosslinking reaction rate and low rubber elasticity.

Next, each of the rubber compositions was tested for crosslinked rubber properties.

Each of the rubber compositions of Examples 1, 3, 4 and 6 and Comparative Examples 1, 2, 3 and 5 was crosslinked using a vulcanizing press set such that the crosslinking temperature was constant at 175° C. and the vulcanization time was 1.5 times as long as each tc90.

The resultant crosslinked rubber, as a test sample, was subjected to physical testing for tensile properties and rubber hardness in accordance with JIS K6251 and JIS K6253.

The crosslinked rubber was also tested for elution in water (potassium permanganate consumption).

More specifically, a crosslinked rubber sheet with a thickness of 2 mm was punched into two circular sheets, one with a diameter of 4.5 cm and the other with a diameter of 3.6 cm. These samples were subjected to an extraction treatment with 160 mL of pure water at 80° C. for six hours, and the resultant water, as a test sample, was measured for potassium permanganate consumption in accordance with JIS T9010

The respective test results are shown in Table 3.

TABLE 3

| | | | | | | | |
|---|---|---|---|---|---|---|---|
| | | | | Comparative | Comparative | Comparative | Comparative |
| Example 1 | Example 3 | Example 4 | Example 6 | Example 1 | Example 2 | Example 3 | Example 5 |

Vulcanized rubber properties

| Vulcanization | Temperature [° C.] | 175 | | | | | | | |
|---|---|---|---|---|---|---|---|---|---|
| condition | Time [min.] | 13.1 | 5.4 | 6.5 | 3.8 | 15.0 | 31.1 | 31.4 | 3.8 |

| | | | | | | | | |
|---|---|---|---|---|---|---|---|---|
| Rubber hardness [JIS-A] | 42 | 40 | 41 | 39 | 38 | 38 | 41 | 37 |
| 200% intermediate stress [MPa] | 2.2 | 2.0 | 1.8 | 1.7 | 1.6 | 1.7 | 2.0 | 1.5 |
| Fracture stress [MPa] | 3.8 | 3.3 | 3.6 | 3.3 | 4.0 | 4.5 | 3.7 | 3.5 |
| Fracture elongation [%] | 347 | 345 | 378 | 398 | 457 | 496 | 378 | 443 |

Elution in water

| | | | | | | | | |
|---|---|---|---|---|---|---|---|---|
| Potassium permanganate | 0.0 | 0.2 | 0.2 | 0.2 | 0.4 | 0.2 | 0.2 | 0.4 |
| consumption [mg/L] | | | | | | | | |

As shown in Table 3, in all Examples, rubber hardness and intermediate stress (200%) were higher than those in Comparative Example 1 using a conventional crosslinking agent (BSH), which is an indication of high rubber elasticity.

It was also shown that the rubber obtained in Examples had less elution in water than in Comparative Example 1 using a conventional crosslinking agent (BSH).

As described above, it was proved that as compared to conventionally used 6-(dibutylamino)-1,3,5-triazine-2,4-dithiol (BSH), the primary aminotriazine dithiol compound represented by Formula (1) above, when contained in a chlorinated butyl rubber composition as a crosslinking agent, served to crosslink the composition at a faster rate, and the resultant rubber product had high rubber elasticity and less elution of additives.

1. A crosslinking agent for chlorinated butyl rubber represented by Formula (1) below: 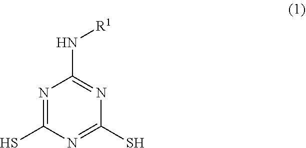
where R¹ is a linear or branched hydrocarbon group having 6 to 10 carbon atoms.

2. A rubber composition comprising a compound represented by Formula (1) below in an amount of 0.01 to 10 parts by weight per 100 parts by weight of chlorinated butyl rubber: 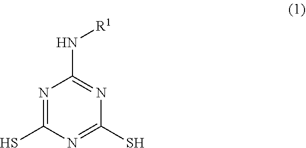
where R¹ is a linear or branched hydrocarbon group having 6 to 10 carbon atoms.

3. A rubber product obtained by crosslinking the rubber composition according to claim 2.

4. A medical rubber composition comprising the rubber composition according to claim 2.

5. A medical rubber product obtained by crosslinking the medical rubber composition according to claim 4.
